(19) United States
(12) Patent Application Publication
(10) Pub. No.: US 2009/0318890 A1
(43) Pub. Date: Dec. 24, 2009

(54) Portable Medical Foam Apparatus

(76) Inventor: Frank Levy, Fort Meyers, FL (US)

Correspondence Address: ROBERT M. SCHWARTZ, P.A., P.O. BOX 221470, HOLLYWOOD, FL, 33022, US

(21) Appl. No.: 12/210,368

(22) Filed: Sep. 15, 2008

Related U.S. Application Data
(63) Continuation-in-part of application No. 11/945,674, filed on Nov. 27, 2007, now Pat. No. 7,543,760.
(60) Provisional application No. 60/867,323, filed on Nov. 27, 2006.

(51) Int. Cl. A61M 5/155; B67D 5/54

(52) U.S. Cl. 604/508; 222/145.5; 604/28; 604/140; 222/373

(57) ABSTRACT

An apparatus for producing medical foam having a foam generation unit including a fluid reservoir, a fluid delivery line and a foam generation tip; and a compressed gas unit including at least one container of compressed gas, a source of electric power, and a gas regulator valve.

### INDEX TO RELATED APPLICATIONS

This application is a continuation-in-part of U.S. patent application Ser. No. 11/945,674 filed Nov. 27, 2007 which claims the benefit of U.S. Provisional Patent Application Ser. No. 60/867,323 filed Nov. 27, 2006 the disclosures of which are incorporated herein by reference in their entirety.

### BACKGROUND OF THE INVENTION

The present invention utilizes Venturi effect to produce the foams and protect them from the apparatus. The present invention is simple to manufacture and use because it does not require an impeller and incorporated fan with a foam generator in order to create foam/foams and dispense them from the apparatus.

The Venturi effect is an example of Bernoulli's principle, in the case of incompressible fluid flow through a tube or pipe with a constriction in it. The fluid velocity must increase through the constriction to satisfy the equation of continuity, while its pressure must decrease due to conservation of energy: the gain in kinetic energy is supplied by a drop in pressure or a pressure gradient force.

The limiting case of the Venturi effect is choked flow, in which a constriction in a pipe or channel limits the total flow rate through the channel because the pressure cannot drop below zero in the constriction. Choked flow is used to control the delivery rate of water and other fluids through spigots and other valves plus portable CO2 delivery system. The portable apparatus of the present invention utilizes a source of compressed gas to produce the desired pressure and airflow for the effective creation of medical foam.

### BRIEF SUMMARY OF THE INVENTION

The present invention provides for a novel apparatus for producing medical foam.

In one embodiment the present invention is an apparatus for producing medical foam comprising:
(a) a foam generation unit having a fluid reservoir, a fluid delivery line and a foam generation tip;
(b) a compressed gas unit having at least one container of compressed gas, a source of electric power, and the gas regulator valve.

The fluid reservoir contains the aforementioned medical solution. The compressed gas is any suitable compressed gas. Suitable compressed gases may include carbon dioxide, atmospheric air, nitrogen, helium, or mixtures thereof. The compressed gas is contained in one or more compressed gas containers.

The apparatus has source of electric power that may be delivered by batteries providing between about 3-24 volts.

The apparatus has a foam generation tip that includes a membrane providing a surface for the formation of medical foam/foams. In a preferred embodiment, the gas regulator valve is an electronically activated solenoid.

Additionally preferred, the gas regulator valve is an electronically activated solenoid controlled by a pressure activation switch or actuator.

The pressure switch activates the solenoid when depressed.

In one embodiment, the present invention utilizes an air delivery system whereby the air is delivered by compressed gas. Any compressed gas can be used. Preferably, the compressed gas is selected from compressed ambient air, carbon dioxide, nitrogen, helium, oxygen, or combinations thereof.

In one embodiment, the apparatus of the present invention includes compressed air storage, with a hose or other acceptable transport mechanism to deliver the compressed gas to the foam generation tip or any other receptacle.

The foam generation tip includes a novel arrangement by which compressed air enters the first end of the foam generation tip, the interior of the foam generation tip as an inlet for providing medical solution to the tip, and the pressure produced within the foam generation tip draws its solution from the inlet into the interior of the tip. The compressed air continues to travel towards the second end of the foam generation tip onto which a membrane is affixed. The membrane provides a surface at which the medical solution mixes with the compressed air and the medical solution foams. The compressed air passes through the membrane and lifts the foams off the membrane outward from the foam generation tip. Thus, the solution, now foamed by the compressed gas, can be delivered.

In one embodiment, a user will utilize two separate units of the apparatus wherein a first unit includes at least one compressed air cylinder and a valve for controlling the release of compressed air from the cylinder. In one embodiment the valve for controlling the release of compressed air is an electronic solenoid.

The present invention also relates to methods of medical treatment.

In one embodiment. The invention is a method for providing sclerotherapy comprising the steps of:
(a) providing a portable CO2 apparatus;
(b) providing a container with a scleroscant, said container having an entrance, an exit, and a release means regulating said exit;
(c) attaching a sclerotherapy injection needle to the exit of said container;
(d) attaching a medically acceptable directional device from the apparatus to the entrance of the container with scleroscant;
(e) initiating an actuator of the apparatus to release CO2;
(f) producing a mixture of scleroscant and CO2 in the container;
(g) injecting said needle into a vein;
(h) activating said release means to deliver said mixture to said vein.

In medical uses, CO2 is used because it is safer and has less complications than air or oxygen in the same uses. CO2 diffuses more naturally in body tissues and is absorbed in the body more rapidly and with less side effects.

CO2 is used in foam sclerotherapy, de-compartmentilization of tissues, arteries, veins, and nerves, and radiological imaging, cardiac imaging for evaluation of the vascularity of the heart and surrounding tissues, oncology and urology diagnostics

It is used for imaging by infiltrating the tissues, body cavities, and abdomen for better visualization. The CO2 can also expand internal body cavities and tissues enabling better diagnostic techniques.

In a preferred embodiment, the CO2 gas that is expelled from the invention will ultimately be used for foaming of sclerotherapy solutions for the treatment of varicose and spider veins. These solutions may include, but would not be limited to Sodium Tetradecol, Polyglyconate, Saline, and Glycerine.

CO2 is used to aerate and subsequently cause foam for schlerotherapy solution. CO2, once introduced to a vein, obliterates the lumen of the vein to eliminate the varicose and spider vein problem.

The invention can be used for the delivery of CO2 or other gases into a medical ambu bag system that will ultimately be drawn up through a syringe for forming the sclerotherapy foam.

The ambu bag is a reservoir that is used to house the CO2 that is provided from existing CO2 tanks. The empty bag is filled with CO2 and then the CO2 is drawn up into a syringe for foaming the solutions in sclerotherapy. The invention can also be used to fill directly into a syringe causing the medical solution to be aerated and become foam.

The present invention is portable, compact, and electronic for the use in the field for portable medical uses, military field use, and any other use requiring CO2 for its performance, such as; imaging, tissue separation, and vascular/vessel compartmentalization.

The present invention can also be adapted and used for other gases to be dispensed, under the same nature, such as; oxygen, nitrogen, helium, or any other gas needed to be contained in a compact, portable delivery system.

In a preferred embodiment, the present invention is completely battery operated.

The invention can be used to provide CO2 or like gases to one syringe, two syringes with a stop-cock connector, or any container that can house or store the CO2 before using it in medical devices, such as; sclerotherapy, imaging, differentiation of tissues, arterial/venous/neurological separation.

The invention can deliver CO2 from an adjustable port with control of the PSI from OPSI to 120PSI.

Previous methods utilizing large CO2 tanks and regulators are dangerous because of the risk of a seal, valve, or part malfunction causing a projectile in a medical setting. The present invention is safer as it eliminates this possibility of malfunction.

The invention requires very little space to store, as opposed to the cumbersome existing tank systems and is much easier to use, with a push button actuator to initiate operation.

The present invention is much less expensive then current CO2 tank systems.

Acquisition of the CO2 in the present invention now requires only cartridges which can be delivered in a small box. The current tanks require filling at a filling station which involves the transport of a large quantity of CO2 which could also result in an explosion in the event of a motor vehicle crash.

The invention can be used by filling of the syringe directly, as opposed to the current system which requires the use of a filling bag which can not always eliminate the oxygen that is delivered into the syringe.

Additionally, the filling bag (present technique), as stated above, cannot assure that oxygen does not get into the bag and ultimately end up in the syringe at the time of foam preparation.

### BRIEF DESCRIPTION OF THE DRAWINGS

FIG. 1 is a side perspective view of an apparatus including compressed gas cylinders and a solenoid of the present invention.

FIG. 2 is an alternative embodiment including 12 gram and 16 gram CO2 cartridges.

FIG. 3 is an alternative embodiment including a larger compressed gas cylinder connected to a solenoid of the present invention.

FIG. 4 is a perspective view of the foam generating tip and foam solution reservoir of the modification to the present invention.

FIG. 5 is close-up view of foam generating tip shown in FIG. 4.

### DETAILED DESCRIPTION OF THE PREFERRED EMBODIMENTS

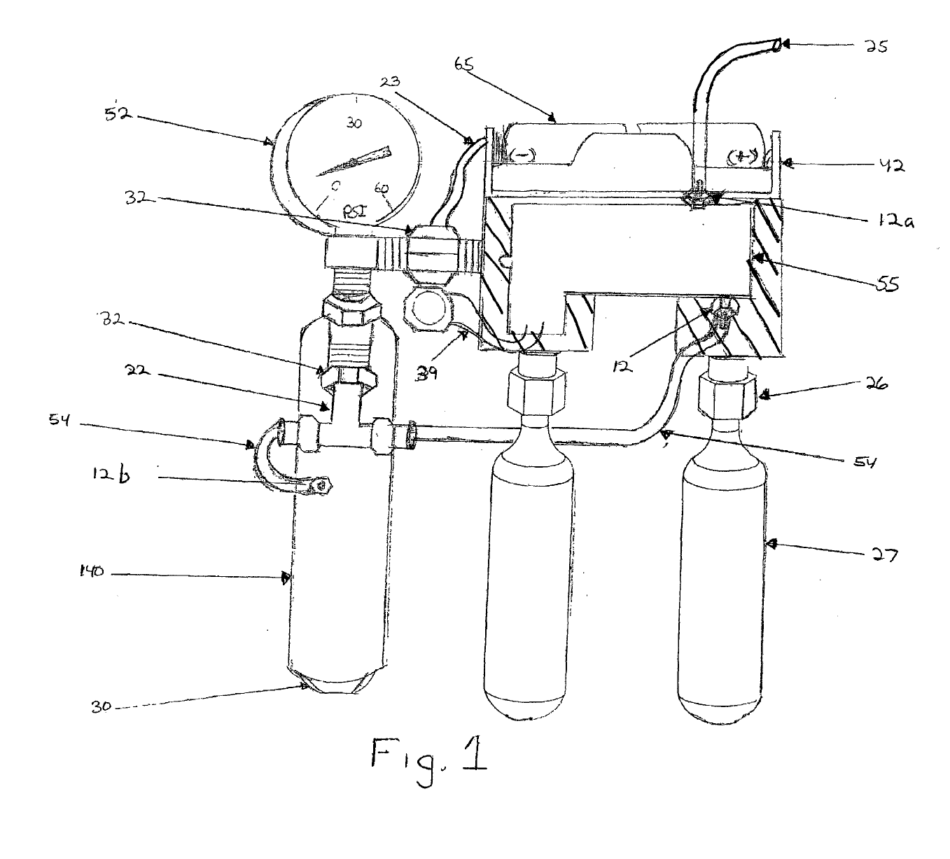

In FIG. 1 compressed gas unit 1 comprises solenoid 55 with at least one compressed gas cylinder. In one embodiment, compressed gas cylinder 27 is 25 g or larger. Compressed gas cylinder 27 is secured into position to unit 1 by means of cylinder cartridge puncture valve 26 and T “puncture” fitting 74.

In a preferred embodiment, cylinder cartridge puncture valve 26 has a mechanism for piercing cylinder 27, as is known, and holding or securing said cylinder in place.

Compressed air enters solenoid 55 from compressed gas cylinder 27 by means of cylinder cartridge puncture valve 26 and T “puncture” fitting 74. Compressed gas unit 1 has at least one battery 65, held in place by battery holder 42, for providing electrical power by which solenoid 55 may be activated and then regulated by pressure activation switch or actuator 37. Battery 65 supplies power to solenoid 55 through battery to switch wire assembly 23, which is secured in place by pressure nut 32. Compressed air unit 1 has an electrical wire 39 for providing necessary electricity to solenoid 55. Unit 1 also comprises black rock regulator 140, which is regulated by secondary regulator adjustment knob 30 when solenoid 55 is activated. Black rock regulator 140 is connected to unit 1 at pressure nut 32 along a threaded mounting. Compressed gas cylinder 27 is secured to unit 1 by cartridge puncture valve 26 as is commonly known. In one embodiment, compressed gas cylinder 27 is a 25 g cylinder. Compressed air leaves black rock regulator 140 by means of a 10/32″ hose port 12b, flows through hose junction 22 by means of ⅛″ pressure hose 54 till reaching the 10/32″ hose port 12b affixed to solenoid 55. From said hose port 12, the compressed air enters solenoid 55. Compressed air unit 1 also has an outlet air port 25, connected to solenoid 55 through intermediate 10/32 hose port 12a, for transporting compressed gas from solenoid 55 in compressed air unit 1 to foam generation unit 2. Outlet gas may be monitored with pressure gauge 52. Unit 1 has battery holder 42 for securing battery 65 into position.

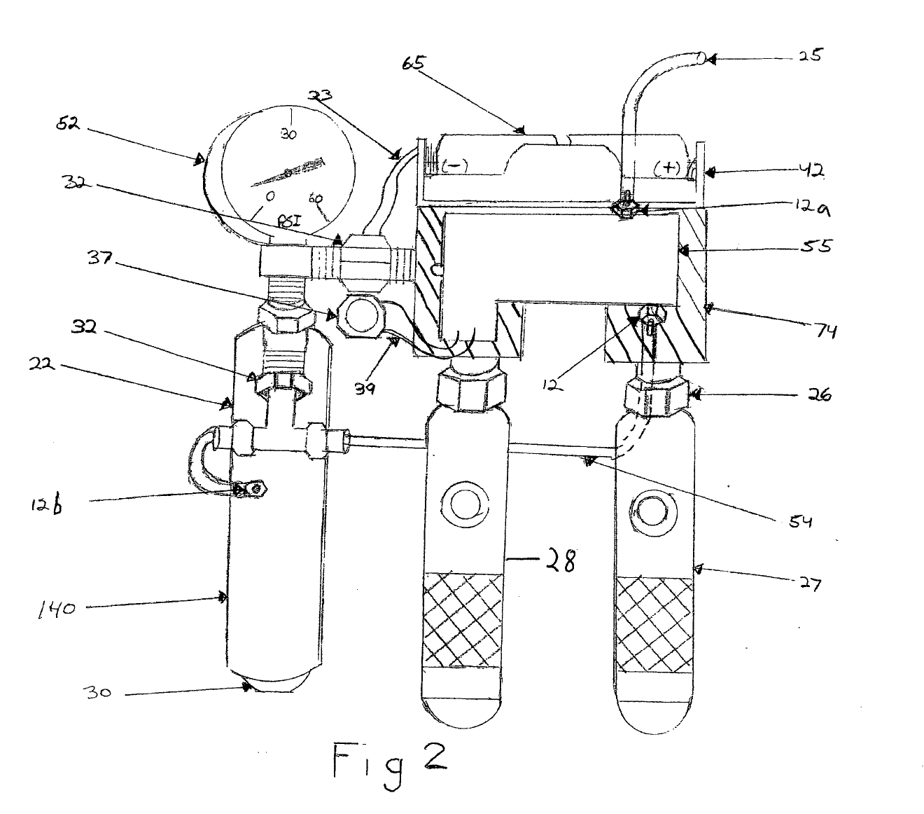

In FIG. 2, one embodiment featuring compressed gas cylinder 28, a 12 g or 16 g compressed gas cylinder, may substitute compressed gas cylinder 27. Housing 74 connects puncture valve 26 with solenoid 55.

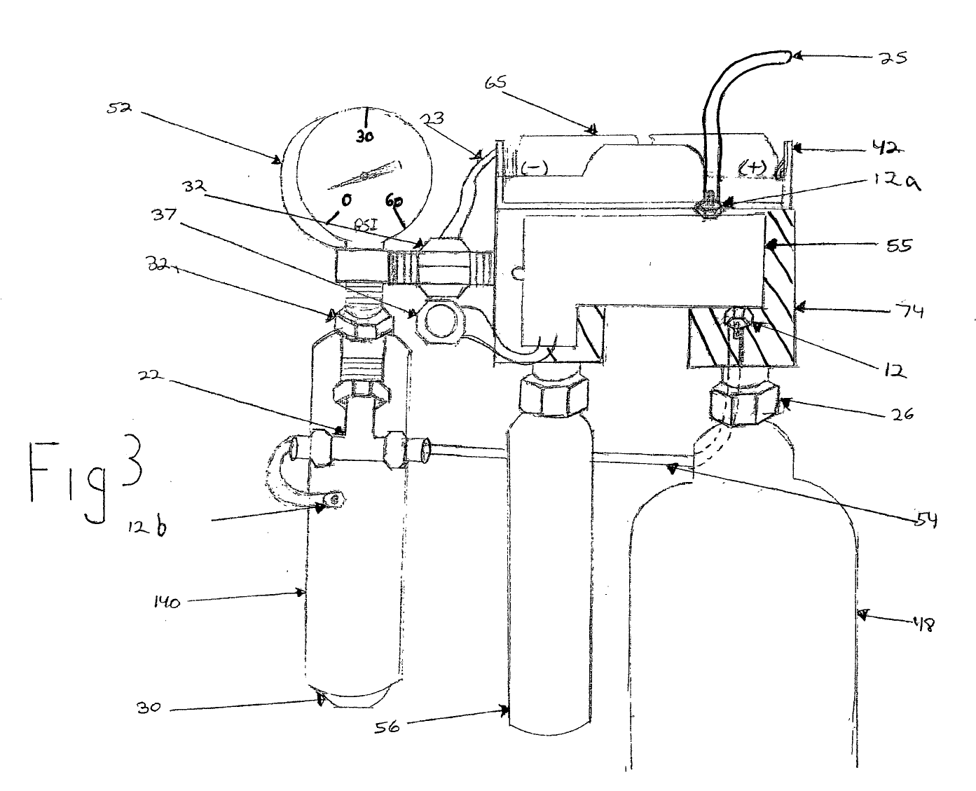

In FIG. 3, another embodiment featuring an accessory tank CO2 delivery system, may substitute compressed gas cylinder 27. Said figure may also substitute a second compressed gas cylinder 27 with expansion chamber 56 used with single compressed gas cylinder 48.

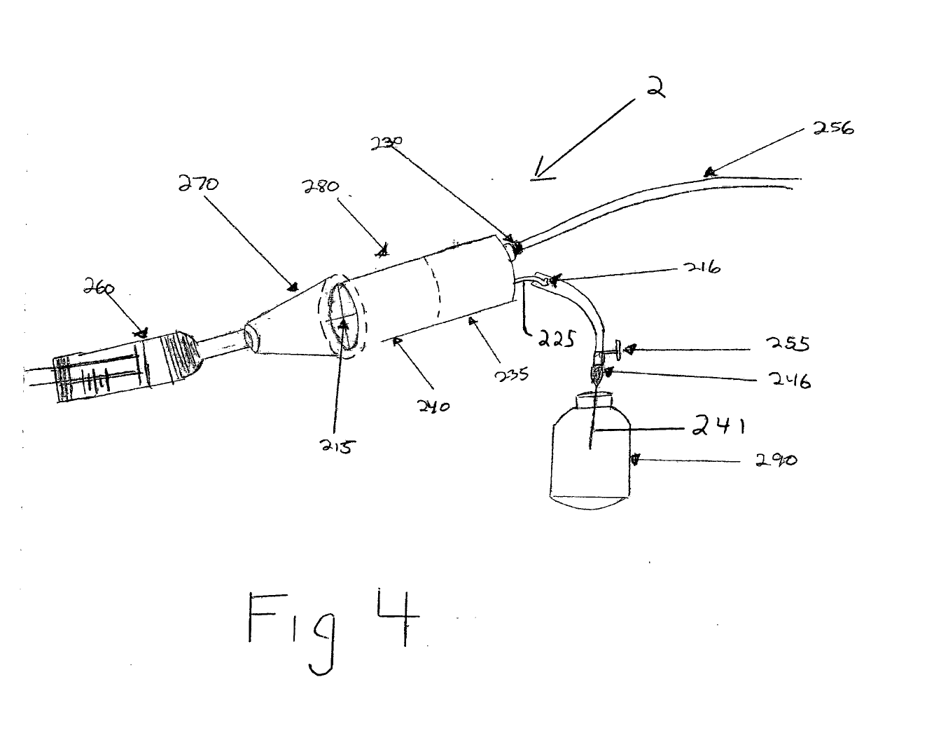

In FIG. 4, unit 2 features micro hose 256, a CO2 delivery system designed to receives compressed gas from Unit 1. Compressed air leaving unit 1 via outlet air port 25 enters unit 2 via micro hose 256. Compressed air passes through air hose inlet 230 and enters foam generation tip 280.

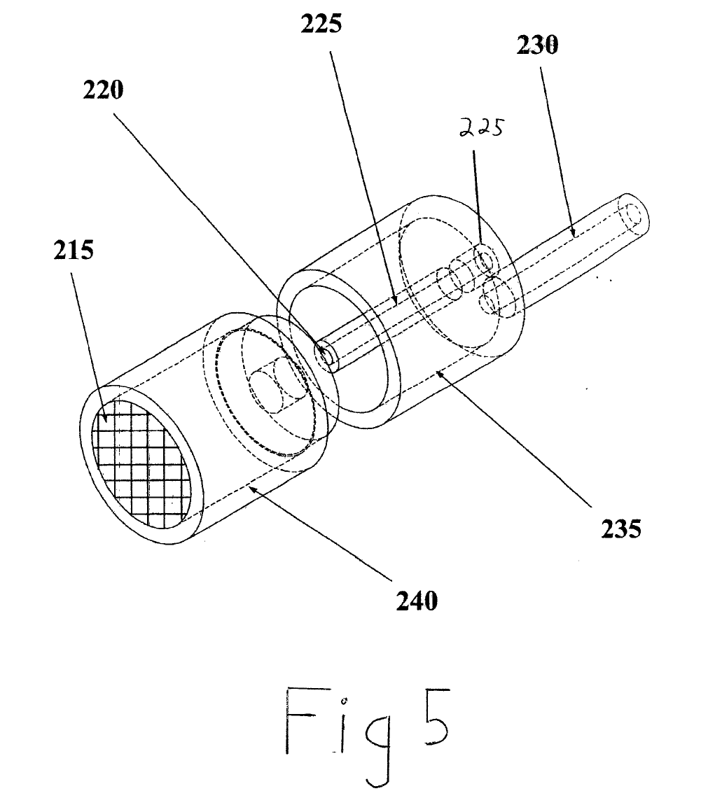

FIG. 5, foam generation tip 280 also includes foam solution delivery line 225 that has outlet 220 for delivering foam solution into upper chamber 240.

In a preferred embodiment, solution from solution reservoir 290 travels up needle 241 and hub 246 when compressed gas enters tip assembly 280 through inlet 256 after being actuated and released from unit 1. Compressed gas entering tip assembly 280 imparts negative pressure on solution in reservoir 290 and draws solution through needle 241 and into tip solution inlet 225 through hose 216. Stop cock 255 is used to regulate or stop flow of solution from reservoir 290. Solution enters solution delivery line 225 from solution line 216. Compressed gas traveling from lower chamber 235 to upper chamber 240 creates negative pressure inside the foam generation tip 280, such that medical foam solution exiting outlet 220 mixes with compressed gas and forms medical foam that forms on membrane 215. The force of the compressed gas traveling through foam generation tip 280 and exiting through membrane 215 lifts medical foam/foams outward from membrane 215 and projects the foam into the foam dispensing port 270. The medical foam then exits tip assembly 280 through syringe hub 260 and is directed as desired.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. An apparatus for producing medical foam comprising:
(a) a portable compressed gas unit having at least one container of compressed gas, a hose for directing said compressed gas, a source of electric power, and a gas regulator valve;
(b) an electronic actuator for initiating and ceasing the flow of said compressed gas;
(c) a medically acceptable directional device for application of said compressed gas;
wherein said apparatus provides compressed gas for medical procedures.

2. The apparatus of claim 1 wherein said compressed gas is a medically acceptable gas.

3. The apparatus of claim 1 wherein said compressed gas is carbon dioxide, atmospheric air, or mixtures thereof.

4. The apparatus of claim 1 wherein said compressed gas is supplied with replaceable compressed gas cartridges.

5. The apparatus of claim 1 wherein said source of electric power is delivered to the unit by batteries.

6. The apparatus of claim 1 wherein said actuator is operable as an electronic push button device.

7. The apparatus of claim 1 wherein said actuator is located directly on the apparatus.

8. The apparatus of claim 1 wherein said actuator is operated and positioned remote from the unit.

9. The apparatus of claim 1 wherein said actuator is operable remotely and attached to the directional device.

10. The apparatus of claim 1 having a foam generation tip with a receiving inlet connected to said hose for directing compressed gas and an outlet from said foam generation tip to said directional device for application of foam formed of aerated solution.

11. A method for providing sclerotherapy comprising the steps of:
(a) providing a portable CO2 apparatus according to claim 1;
(b) providing a container with a scleroscant, said container having an entrance, an exit, and a release means regulating said exit;
(c) attaching a sclerotherapy injection needle to the exit of said container;
(d) attaching a medically acceptable directional device from the apparatus to the entrance of the container with scleroscant;
(e) initiating an actuator of the apparatus to release CO2;
(f) producing a mixture of scleroscant and CO2 in the container;
(g) injecting said needle into a vein;
(h) activating said release means to deliver said mixture to said vein.
